(19) United States
(12) Patent Application Publication
(10) Pub. No.: US 2006/0062865 A1
(43) Pub. Date: Mar. 23, 2006

(54) Tyrosinase inhibitor and method for preparation

(76) Inventors: Nebojsa Ilic, Highland Park, NJ (US); Diego A. Moreno-Fernandez, Highland Park, NJ (US); Ilya Raskin, Manalapan, NJ (US)

Correspondence Address: Diane Dunn McKay, Esq.;Mathews, Collins, Shepherd & McKay, P.A., 100 Thanet Circle, Suite 306, Princeton, NJ, 08540, US

(21) Appl. No.: 10/943,362

(22) Filed: Sep. 17, 2004

(51) Int. Cl. A61K 36/185

(52) U.S. Cl. 424/769

(57) ABSTRACT

A Tyrosinase inhibitor extracted from Osage orange (Maclura pomifera) heartwood is described. Methods of producing the extract as well as a variety of applications for the extract are also described. Applications for the extract include use as a skin-whitening agent in cosmetic products, a pharmaceutical agent interfering with the aromatic amino acid metabolism, an insect controlling agent, and an agent that prevents browning of fruits and vegetables.

### FIELD OF THE INVENTION

A Tyrosinase inhibitor extracted from Osage orange (Maclura pomifera) heartwood is described. Methods of producing the extract as a well as a variety of applications for the extract are also described. Applications for the extract include use as a skin-whitening agent in cosmetic products, a pharmaceutical agent interfering with the aromatic amino acid metabolism, an insect controlling agent, and an agent that prevents browning of fruits and vegetables.

### BACKGROUND OF THE INVENTION

Multifunctional copper-containing enzyme, tyrosinase (E.C. 1.14.18.1; polyphenol oxidase, PPO; tyrosine hydroxylase) is the key enzyme involved in the biosynthesis of melanin. It catalyzes the rate limiting step, the oxidation of the aromatic amino acid tyrosine to 3,4-dihydroxyphenylalanine (DOPA) and subsequently to DOPA-quinone. DOPA-quinone is then converted by multi-step reactions to melanin pigments. Tyrosinase is widely distributed in nature in many organisms with slightly different forms. See, Jimenez M., Chazarra S., Escribano J., Cabanes J. and Garcia-Carmona F.: Competitive inhibition of mushroom tyrosinase by 4-substituted benzaldehydes, J. Agric. Food Chem. 2001, 49, 4060-4063.

In plants, tyrosinase induced pigmentation is responsible for browning of wounded plant parts. This results in huge economical losses because it shortens the shelf life and decreases the quality of raw fruits and vegetables during post-harvest handling and processing. See, Perez-Gilabert M. and Garcia-Carmona, F.: Characterization of catecholase and cresolase activities of eggplant polyphenol oxydase, J. Agric. Food Chem. 2000, 48, 695-700; and, Kim Y. M., Yun J., Lee C-K., Lee H., Min K R. and Kim Y.: Oxyresveratrol and hydroxystilbene compounds—inhibitory effect on tyrosinase and mechanism of action, J. Biological Chem. 2002, 277(18) 16340-16344.

In insects, the enzyme is involved in defensive and developmental processes and for this reason the tyrosinase inhibitors could be used as insect-control agents. See, Kramer K. J., and Hopkins T. L.: Tyrosine metabolism for insect cuticle tanning, Arch. Insect Biochem. Physiol. 1987, 6, 279-301.

In mammals, the final product of tyrosinase activity is melanin, a pigment responsible for the skin and hair color. It is produced in melanocytes, cells located in the basal layer of the dermis as described in Spritz, R. A. and Hearing, V. J. Jr., Genetic disorders of pigmentation, Adv. Hum. Gent. 1994, 22, 1-45. The concentration and distribution of melanin in epidermis determines our skin color as described in Perez-Bernal A., Munoz-Perez, M. and Camacho, F., Management of facial hyperpigmentation, Am. J. Clin. Dermatol. 2000, 1, 261-268.

Accumulation of high levels of melanin (hyperpigmentation) causes a variety of dermatologic disorders. These include sites of actinic damage (caused by solar UV irradiation), melasma, freckles, age spots as described in Curto E. V., Kwong C., Hermersdoerfer H., Glatt H., Santis C., Virador V., Hearing V. J. and Dooley T. P., Inhibitors of mammalian melanocyte tyrosinase: in vitro comparisons of alkyl esters of gentisic acid with other putative inhibitors, Biochem. Pharmacology 1999, 57, 663-672.

A number of treatments exist for these conditions and the most prominent ones function as inhibitors of tyrosinase activity. Among them the most widely used are 1,4-dihydroquinone and kojic acid. See, Perez-Bernal A., Munoz-Perez, M. and Camacho, F.: Management of facial hyperpigmentation, Am. J. Clin. Dermatol. 2000, 1, 261-268.

Dihydroquinone has been proven to be cytotoxic and to induce the mutagenesis and because of the safety concerns is being phased out as a treatment. Kojic acid is widely used as a tyrosinse inhibitor and skin-whitening agent in cosmetic products as described in Perez-Bernal A., Munoz-Perez, M. and Camacho, F., Management of facial hyperpigmentatio, Am. J. Clin. Dermatol. 2000, 1, 261-268.

Despite this there is a growing demand for new, natural, effective and safe tyrosinase inhibitors.

### SUMMARY OF THE INVENTION

The present invention is directed toward the solvent extract of Osage orange (Maclura pomifera) heartwood. A range of solvents can be used for the extraction. Non-limiting limiting examples of such solvents include acetone, ethyl acetate, acetonitrile, hexane, water, and alcohols such as ethanol, methanol, and propanol. Ethanolic extract is a preferred embodiment of this invention. This extract strongly inhibits tyrosinase enzyme and is easily prepared by extraction of the heartwood, a common tree that grows in the United States. The inhibitory activity of the extract is comparable to that of the commercial skin whitening standard, kojic acid. The extract can also be prepared by extraction of the roots and bark.

The extract is suitable for various applications and may be used as an effective skin-whitening agent in cosmetic products, a pharmaceutical agent interfering with the aromatic amino acid metabolism, an insect controlling agent and an agent that prevents browning of fruits and vegetables.

### BRIEF DESCRIPTION OF THE DRAWINGS

FIG. 1 is a fingerprint profile of the ethanolic extract of Osage orange (Maclura pomifera) heartwood from analysis by Liquid Chromatography-Mass Spectrometry (LC-MS).

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

A novel tyrosinase inhibitor is disclosed. The tyrosinase inhibitor is extracted from Osage orange (Maclura pomifera) heartwood. The extract demonstrates excellent inhibitory activity in mushroom tyrosinase in vitro assay. The strong tyrosinase inhibitory activity enables the Osage orange extract to be used in a variety of applications in which tyrosinase inhibition is sought. Non-limiting examples of such applications include the use as a skin whitening agent in cosmetic products, as a pharmaceutical agent interfering with the aromatic amino acid metabolism, as an insect controlling agent, and in the prevention of browning in fruits and vegetables. More specifically, in a cosmetic application, the extract can be incorporated, for example, in creams, lotions, soaps, shampoos, emulsions, solutions, and other vehicles generally used in cosmetics. As a pharmaceutical agent interfering with the aromatic amino acid metabolism, the extract may be incorporated into a topical application medium. As an insect controlling agent, the extract can be applied anywhere insect control is desired. In the prevention of browning in fruits and vegetables, application may be accomplished by spraying or dipping the fruits and vegetables in the extract.

Osage orange or bow wood (Maclura pomifera, Moraceae family) is a tree native to the southern United States (Arkansas and Texas). The name of tree comes from Osage Indian tribe that lived in home area of the tree and the aroma of its ripe fruit that reminds of orange. In 19th century the tree was widely used by settlers to make living fences because of the sharp thorns present on the branches. That was the way tree was introduced in other parts of the United States. The wood of Osage orange is highly prized for its strength and elasticity, so it is used to make the best bows for archery. It is also very resistant to rotting and termites.

The following example illustrates one process for performing an extraction to obtain the tyrosinase inhibitor.

### EXAMPLE 1

Plant Material: The plant material (heartwood planks) was purchased from commercial suppliers. The Osage orange wood planks were cut and milled into sawdust.

Extraction procedure. The sawdust was extracted in 95% ethanol (1:20 w/v) for 24 hours on the shaker at room temperature. The obtained plant extracts was then filtered (Whatman filter paper No. 1) and dried by rotary evaporation. The dry plant extracts were redissolved in 95% ethanol at appropriate concentrations for the enzyme assay.

Enzyme assay. The mushroom tyrosinase was used for the bioassay. It was purchased from Sigma Chemical Co. (St. Louis, Mo.). The assay was performed by adding 200 microliters of reaction cocktail (mixture of potassium phosphate buffer 50 mM, pH 6.5, 10 ml; 1 mM tyrosine solution, 10 ml and deionized water, 9 ml) and 10 microliters of tested extract dissolved in ethanol at concentration of 1 mg/ml followed by 10 microliters of tyrosinase (1000 units/ml) to 96-well plate. After 2 min incubation at room temperature, the initial absorbance was read at 492 nm on the plate reader. Final absorbance values were read after 10 minutes. The enzyme activity was calculated from the differential values for absorbance and then converted into inhibitory activity. Kojic acid was used as standard at a concentration of 1 mg/ml dissolved in 95% ethanol. The inhibitory activity of kojic acid at 1 mg/ml was 88%.

Analytical Analysis, LC-MS Gradient:

Extract was separated on a Phenomenex® Luna C-8 reverse phase column, size 150×2 mm, particle size 3 μm, pore size 100 Å, equipped with a Phenomenex® SecurityGuard™ pre-column using High Pressure Liquid Chromatograpy (Waters™ dual pump system). The mobile phase consisted of 2 components: Solvent A (0.5% ACS grade acetic acid in double distilled de-ionized water, pH 3-3.5), and Solvent B (100% Acetonitrile). The mobile phase flow was adjusted to a rate of 0.25 ml/min, and a gradient mode was used for all analyses as follows: 0-35 min 95% A-5% A; 35-40 min 5% A; 40-45 min 5% A -95% A.

The results were as follows:

Ethanolic extract of Osage orange (Maclura pomifera) heartwood was tested in vitro for the inhibition of mushroom tyrosinase enzyme. At the concentration of 1 mg/ml the ethanolic extract demonstrated high inhibitory activity against the enzyme. The activity was slightly more potent then commercial compound kojic acid that was used as a control. The inhibitory level for the extract was 91% compared to 88% for kojic acid (Table 1).

TABLE 1
Inhibitory effect of PMI-3047 on the in vitro
tyrosinase(mushroom) enzyme activity.

| Sample | Inhibition at 1 mg/ml |
|---|---|
| PMI-3047 | 91% |
| Kojic acid | 88% |

An investigation of other parts of the plant to determine the inhibitory activity against tyrosinase. The extracts of roots and bark, prepared in the same manner, were tested in assay (Table 2). The extract of heartwood exhibited the highest inhibitory activity (84%), followed by root (66%) and bark extract (26%). While not bound by theory, it appears likely that the tyrosinase inhibitory compounds are present in all plant parts but at different concentrations, with heartwood being the most concentrated source of plant.

TABLE 2
Inhibitory effect of different plant parts on the
in vitro tyrosinase(mushroom) enzyme activity.

| Sample | Inhibition at 1 mg/ml |
|---|---|
| Heartwood | 84% |
| Root | 66% |
| Bark | 26% |
| Kojic acid | 86% |

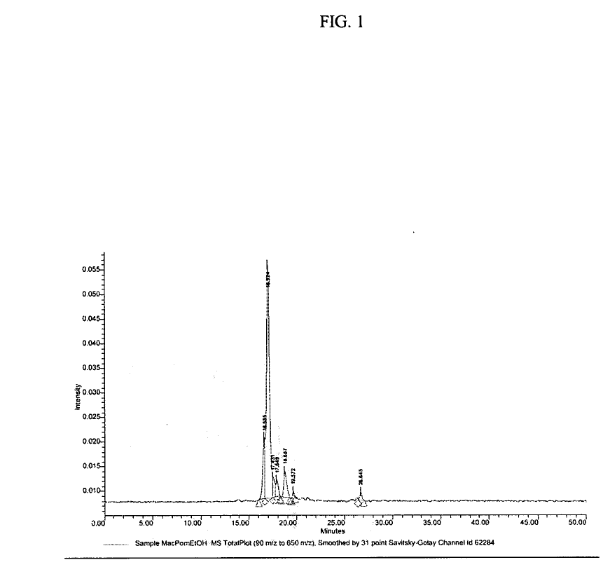

FIG. 1 illustrates the fingerprint profile of the extract by LC-MS. The profile indicates that the extract is a mixture of several compounds. The inhibitory activity of particular single most active compound from the extract may demonstrate much higher inhibitory activity against the enzyme than the whole ethanolic extract.

Various modifications and variations of the described compositions and methods of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described compositions and modes for carrying out the invention, which are obvious to those skilled in the art or related fields.

What is claimed is:

1. A tyrosinase inhibitor comprising
extract of Osage orange (Maclura pomifera) plant material.

2. The tyrosinase inhibitor of claim 1 wherein the extraction solvent is ethanol.

3. The tyrosinase inhibitor of claim 1 wherein the plant material is heartwood.

4. The tyrosinase inhibitor of claim 1 wherein the plant material is roots and/or bark.

5. A method of producing a tyrosinase inhibitor comprising the step of:
extracting Osage orange (Maclura pomifera) heartwood with a solvent.

6. The method of producing a tyrosinase inhibitor of claim 5 wherein the solvent is ethanol.

7. The method of producing a tyrosinase inhibitor of claim 5 wherein the plant material is heartwood

8. The method of producing a tyrosinase inhibitor of claim 5 wherein the extraction comprises the following steps:
extracting the plant material in 95% ethanol in a shaker at about room temperature; and,
filtering the extract.

9. The method of producing a tyrosinase inhibitor of claim 8 further comprising the step of:
drying the filtered extract.

10. A method of inhibiting tyrosinase activity comprising the step of:
applying extract of Osage orange (Maclura pomifera) heartwood to an area in which tryosinase inhibition is sought.

11. The method of claim 10 wherein the area is the skin of a human.

12. The method of claim 10 wherein the extract of Osage orange (Maclura pomifera) heartwood is incorporated into a cosmetic or pharmaceutical product prior to application.

13. The method of claim 10 wherein the area is a surface of a fruit or vegetable.
